(19) United States
(12) Patent Application Publication
(10) Pub. No.: US 2020/0214899 A1
(43) Pub. Date: Jul. 9, 2020

(54) SUPERABSORBENT LAMINATE DRESSING

(71) Applicant: KCI LICENSING, INC., San Antonio, TX (US)

(72) Inventors: Christopher B. Locke, Bournemouth (GB); Timothy M. Robinson, Blandford Forum (GB)

(21) Appl. No.: 16/723,621

(22) Filed: Dec. 20, 2019

Related U.S. Application Data
(60) Provisional application No. 62/788,036, filed on Jan. 3, 2019.

(51) Int. Cl. A61F 13/02; A61M 1/00

(52) U.S. Cl. CPC A61F 13/022; A61F 13/0289; A61F 13/0209; A61F 2013/00119; A61M 1/009; A61M 2207/00; A61F 13/0216

(57) ABSTRACT

A dressing consists substantially of a hydrophilic membrane layer, a superabsorbent material positioned on the hydrophilic membrane layer, and a film layer coupled to the hydrophilic membrane layer and configured to confine the superabsorbent material between the film layer and the hydrophilic membrane layer. The film layer has a high moisture vapor transfer rate.

### CROSS-REFERENCE TO RELATED APPLICATIONS

This application claims the benefit of priority to U.S. Provisional Application No. 62/788,036, filed on Jan. 3, 2019, which is incorporated herein by reference in its entirety.

### BACKGROUND

The present disclosure relates to dressings for treating wounds. Many wounds exude fluid (e.g., blood, pus, etc.). Dressings for such wounds may include absorbent materials or other features that attempt to manage the fluid, for example with the goal of absorbing all fluid from a wound.

Conventional absorbent dressings are bulky, which may create difficulty in applying the dressings and inconvenience for patients. To address these issues, some dressings may include superabsorbent materials which swell when absorbing fluid. However, structures surrounding the superabsorbent materials in such dressings often create physical barriers which restrict the swelling of the superabsorbent materials, thereby reducing the fluid capacity of the superabsorbent materials and reducing the ability of the dressing to manage the fluid exuded from a wound.

Accordingly, a need exists for a flexible, conformable, non-bulky, high-fluid-capacity dressing for wound treatment.

### SUMMARY

One embodiment of the present disclosure is a dressing. The dressing consists substantially of a hydrophilic membrane layer, a superabsorbent material positioned on the hydrophilic membrane layer, and a film layer coupled to the hydrophilic membrane layer and configured to confine the superabsorbent material between the film layer and the hydrophilic membrane layer. The film layer has a high moisture vapor transfer rate.

In some embodiments, the superabsorbent deposits are configured to expand when the superabsorbent deposits absorb fluid. In some embodiments, the film layer is elastic and configured to stretch to allow the superabsorbent material to expand towards the film layer. In some embodiments, the hydrophilic membrane layer is configured to substantially restrict expansion of superabsorbent material towards the hydrophilic membrane layer.

In some embodiments, the superabsorbent material includes a plurality of superabsorbent deposits separated from one another. In some embodiments, the plurality of superabsorbent deposits is formed by printing a slurry of the superabsorbent material in a pattern on the hydrophilic membrane layer. In some embodiments, the plurality of superabsorbent deposits are formed by printing a paste in a pattern on the hydrophilic membrane layer. The paste includes the superabsorbent material and isopropyl alcohol.

In some embodiments, the hydrophilic membrane layer is thermo-bonded to the high moisture vapor transfer rate layer. In some embodiments, the hydrophilic membrane layer is configured to contact a wound and substantially prevent adherence of the dressing to the wound.

In some embodiments, the hydrophilic membrane layer or the film layer comprises an adhesive border couple to a periwound area around the wound.

Another implementation of the present disclosure is a dressing. The dressing includes a first film layer, a second film layer coupled to the first film layer, a superabsorbent material positioned between the first film layer and the second film layer, and a fusible fiber bonding the first film layer to the second film layer and securing the superabsorbent material therebetween.

In some embodiments, the first film layer and the second film layer include a hydrophilic microporous film. In some embodiments, the microporous film has a thickness of approximately 125 microns and includes perforations having diameters between approximately 0.1 microns and 0.2 microns. In some embodiments, the microporous film is configured to allow fluid to flow therethrough and substantially prevent the superabsorbent material from flowing therethrough. In some embodiments, the microporous film is configured to substantially prevent the flow of bacteria therethrough.

In some embodiments, the first film layer is a microporous film layer and the second film layer is a polyurethane drape. In some embodiments, the dressing is sealable over a wound bed and coupled to a pump, the pump configured to draw a negative pressure at the wound bed.

Another implementation of the present disclosure is a method for manufacturing a dressing. The method includes manufacturing a superabsorbent laminate by printing a plurality of superabsorbent deposits on a first film layer and coupling the first film layer to a second film layer to substantially confine the superabsorbent deposits between the first film layer and the second film layer. The method also includes coupling the superabsorbent laminate to at least one of a wound contact layer, an absorbent layer, a manifolding layer, or an antimicrobial layer.

In some embodiments, the method includes forming the first film layer from a microporous film configured to allow water to flow therethrough and substantially prevent the superabsorbent material from flowing therethrough. In some embodiments, the method includes forming the first film layer from a hydrophilic membrane material and forming the second film layer from an elastic material having a high moisture vapor transfer rate.

In some embodiments, the method includes providing a pump and a tube and preparing the dressing to be fluidly coupled to the pump via the tube. The pump is operable to draw a negative pressure at the dressing. The method includes creating a channel through the superabsorbent laminate to facilitate the flow of air therethrough.

In some embodiments, printing the plurality of superabsorbent deposits on the first film layer includes depositing a slurry of superabsorbent material in a pattern on the first film layer. In some embodiments, printing the plurality of superabsorbent deposits on the first film layer includes depositing a paste in a pattern on the first film layer. The paste includes the superabsorbent material and isopropyl alcohol. In some embodiments, coupling the first film layer to the second film layer includes thermo-bonding the first film layer to the second film layer.

In some embodiments, coupling the first film layer to the second film layer includes positioning a fusible fiber layer between the first film layer and the second film layer and fusing the fusible fiber layer to the first film layer and the second film layer.

### BRIEF DESCRIPTION OF THE DRAWINGS

FIG. 1 is a perspective view of a first embodiment of a superabsorbent laminate, according to an exemplary embodiment.

FIG. 2 is an exploded view of a second embodiment of a superabsorbent laminate, according to an exemplary embodiment.

FIG. 3 is an exploded view of a third embodiment of a superabsorbent laminate, according to an exemplary embodiment.

FIG. 4 is an exploded view of a fourth embodiment of a superabsorbent laminate, according to an exemplary embodiment.

### DETAILED DESCRIPTION

Referring generally to the FIGURES, various embodiments of a superabsorbent laminate are shown, according to exemplary embodiments. Each embodiment may be configured for use as a stand-alone dressing (e.g., without additional dressing layers), or may be incorporated with other layers in a dressing or other wound treatment system. In some embodiments, the superabsorbent laminates described herein may be used with negative pressure wound therapy. The superabsorbent laminates disclosed herein improve existing dressings by being highly conformable, flexible, and articulable, by providing enhanced absorption capacity and evaporation rates, by being less bulky than existing dressings, and by providing an indication to a user of the level of fluid absorption based on the swelling of a superabsorbent, among other advantages.

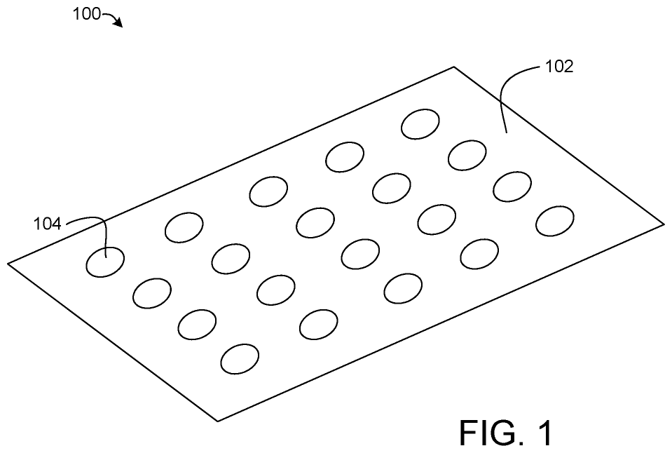

Referring to FIG. 1, a first superabsorbent laminate 100 is shown, according to an exemplary embodiment. The first superabsorbent laminate 100 includes a film layer 102 and multiple superabsorbent deposits 104. The first superabsorbent laminate 100 is configured to draw fluid through the film layer 102 for absorption by the superabsorbent deposits 104. In some embodiments, the fluid may then evaporate to the environment from the superabsorbent deposits 104.

The film layer 102 may be a hydrophilic membrane, for example a highly hydrophilic membrane. The film layer 102 may include a microporous film, for example made of polyvinylidene difluoride (PVdF) and, in some embodiments, treated to be hydrophilic. The film layer 102 may include perforations that are large enough to draw water molecules through the film layer 102 while also small enough to prevent a superabsorbent material of the superabsorbent deposits 104 from passing through the perforations. For example, the perforations may be between approximately 0.1 microns and 0.2 microns in diameter. The film layer 102 may have a thickness of approximately 125 microns. In various embodiments, the film layer 102 may include a 0.45-micron-pore-size nylon membrane sold under the tradename Magna™ by GVS, a PVdF 0.22-micron-pore-size membrane sold under the tradename Durapore® by Merck, or a 0.7-micron-pore-size GF/F grade glass microfiber sold under the tradename Whatman® by GE Healthcare Life Sciences.

The film layer 102 may also substantially prevent bacteria from passing therethrough in either direction. In some embodiments, a wound-facing side of the film layer 102 may be coated (provided) with an antibacterial substance such as charcoal to neutralize bacteria at the film layer 102.

The first superabsorbent laminate 100 includes multiple superabsorbent deposits 104 arranged on a top surface of the film layer 102. As shown in FIG. 1, the superabsorbent deposits 104 are arranged in an array (four rows of five superabsorbent deposits 104). In various embodiments, various numbers, arrangements, patterns, etc. of superabsorbent deposits 104 are possible. For example, in some embodiments the superabsorbent deposits 104 are arranged in concentric circles. As another example, the superabsorbent deposits 104 may be unevenly spaced around the dressing. As another example, the superabsorbent deposits 104 may be positioned in some areas of the first superabsorbent laminate 100 to direct fluid to or provide absorption at those areas, while other areas of the first superabsorbent laminate 100 may be free of superabsorbent deposits 104 to prevent fluid from being directed to or absorbed at such deposit-free areas. As another example, the density of the superabsorbent deposits 104 may be varied to vary the absorbent capacity of the first superabsorbent laminate 100. In some embodiments, different types of superabsorbent materials (e.g., rapid absorbers, slow absorbers, high capacity absorbers) are included in superabsorbent deposits 104 positioned in different areas of the first superabsorbent laminate 100 to customize the fluid uptake of the first superabsorbent laminate 100. It should be understood that the arrangement of the superabsorbent deposits 104 is highly configurable and may be customized for various types of wounds and various types of dressings.

The superabsorbent deposits 104 may include one or more of various superabsorbent materials, for example acrylamide, 2-acrylamido-2-methylpropanesulfonic acid, sodium polyacrylate (e.g., as sold under the tradename Luquasorb® 1161 by BASF), polyacrylamide copolymer, ethylene maleic anhydride copolymer, cross-linked carboxymethylcellulose, polyvinyl alcohol copolymers, cross-linked polyethylene oxide, etc. In some embodiments, the superabsorbent material is reduced to a fine powder and combined with a mix of water and isopropyl alcohol (IPA) to form a paste (e.g., with a superabsorbent:water:IPA ratio of approximately 1:15:6). The paste may take up to 60 minutes to fully form, and adjustments may be made by adding more IPA to reduce viscosity or adding more water to increase viscosity. The paste may be printed (deposited) on the film layer 102 to form the superabsorbent deposits 104, for example using a 3-axis glue dispenser. In some cases, superabsorbent particles may be plasticized with water to soften the hard particles to a gel or paste which may be readily processed by the dispenser. In some cases, the paste is formed without a carrier polymer, which may increase the activity and concentration of the superabsorbent material. The isopropyl alcohol may act to modify the viscosity of the paste and act as a drying aid. In various embodiments, water-soluble organic solvents of different volatilities (e.g., ketones, ethers, esters) may be used in addition to or instead of IPA to control the drying rate of the superabsorbent deposits 104 and/or suppress swelling of the superabsorbent material to control/reduce paste viscosity.

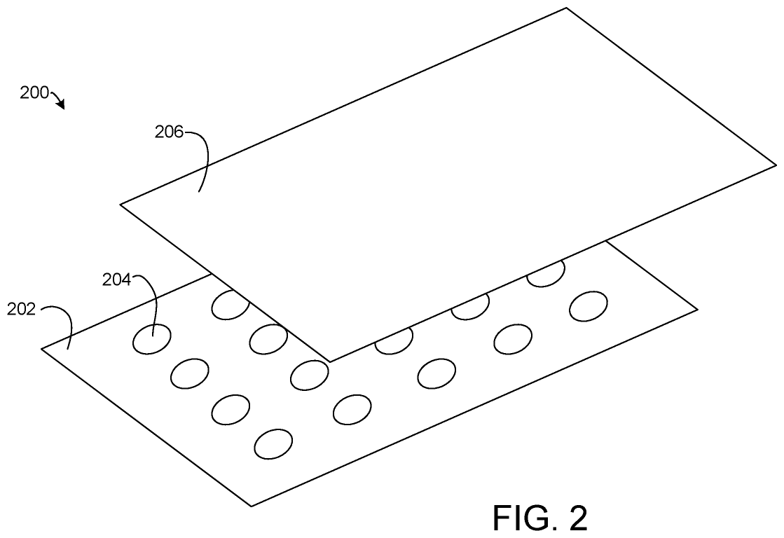

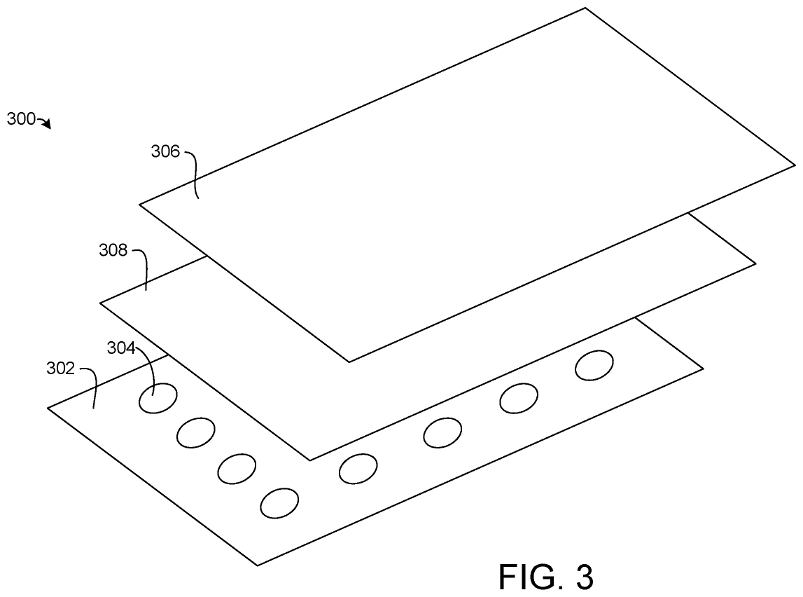

In other embodiments, to form the superabsorbent deposits 104, the superabsorbent material(s) may be formed into a slurry and printed (deposited) on the film layer 102. In some embodiments, the superabsorbent material(s) is formed into a slurry with polyethylene oxide (PEO) to bind the superabsorbent material(s) together. The PEO may be dissolved into a non-aqueous solvent, such as an alcohol (e.g., ethanol) or ketone (e.g., propanone) into which particles of the superabsorbent material(s) may be dispersed, which may vary the absorption or drying rate of the superabsorbent deposits 104. In some embodiments, another water-soluble or swelling carrier may be used instead of or in addition to PEO, for example pyrrolidone and polyvinyl alcohol. Various PEOs may be used to adjust hardness of the superabsorbent deposits 104. In some embodiments, a disintegrant may be added to the slurry or paste to enable a more rapid deployment and increase water absorption rates of the superabsorbent deposits 104. In some embodiments, activated carbon or other ion exchange additives may be added to the slurry or paste, which may increase the absorption capacity of the superabsorbent. In some embodiments, the superabsorbent deposits 104 may adhere to the film layer 102 due to an adherent property of the slurry or paste (i.e., without an added binding agent that may slow absorption). For example, the superabsorbent material may partially penetrate the adjacent surface when swollen, such that when the superabsorbent material dries it remains locked or bonded to the adjacent surface(s)/substrate(s). In other embodiments, the first superabsorbent laminate 100 is incorporated into a dressing that includes one or more additional layers that couple the superabsorbent deposits 104 to the film layer 102, for example as shown in FIGS. 2-3 and described in detail with reference thereto.

In alternative embodiments, the superabsorbent deposits 104 may be formed by coating an uncrosslinked solution of a polymer may be coated onto the film layer 102 and then cross-linked to form a superabsorbent material, for example by exposure to ultraviolet radiation or through a gamma sterilization process.

Referring now to FIG. 2, a second superabsorbent laminate 200 is shown, according to an exemplary embodiment. The second superabsorbent laminate 200 includes a hydrophilic membrane layer 202, superabsorbent deposits 204 positioned on the hydrophilic membrane layer 202, and a top film layer 206 coupled to the hydrophilic membrane layer 202 and configured to confine the superabsorbent deposits 204 between the top film layer 206 and the hydrophilic membrane layer 202. In some embodiments, the hydrophilic membrane layer 202 may be the same as or similar to the film layer 102 of FIG. 1 while the superabsorbent deposits 204 may be the same as or similar to the superabsorbent deposits 104 of FIG. 1, for example such that the second superabsorbent laminate 200 includes the first superabsorbent laminate 100.

The hydrophilic membrane layer 202 is positioned at the “bottom” of the second superabsorbent laminate 200, i.e., at a wound-facing side of the second superabsorbent laminate 200. The film layer 102 may be configured to contact a wound while substantially preventing adherence to the wound. The hydrophilic membrane layer 202 facilitates the flow of fluid therethrough from a wound or other fluid source to the superabsorbent deposits 204.

The superabsorbent deposits 204 are configured to expand (swell, increase in volume) when the superabsorbent deposits 204 absorb fluid. The hydrophilic membrane layer 202 may substantially restrict the expansion of the superabsorbent deposits 204 towards the hydrophilic membrane layer 202. For example the hydrophilic membrane layer 202 may be substantially inelastic. The hydrophilic membrane layer 202 may thereby substantially prevent the swelling of the superabsorbent deposits 204 from exerting pressure on a wound, thereby reducing patient discomfort.

The top film layer 206 may be made of an elastic film having a high moisture vapor transfer rate. The top film layer 206 is configured to stretch to allow the superabsorbent deposits 204 to expand towards the top film layer 206 (i.e., away from a wound) when the superabsorbent deposits 204 absorb fluid and expand. The superabsorbent deposits 204 may thereby be allowed to expand to a substantially maximum size to substantially maximize the fluid capacity of the superabsorbent deposits 204. For example, the top film layer 206 may be made of a thermoplastic polyurethane, for example as sold under the trade name Elasthane™ by DSM or sold under the trade name Dureflex® by Covestro, or copolyamide or copolyester. The top film layer 206 may be between approximately 10 microns and 60 microns thick. The top film layer 206 may be coupled to the hydrophilic membrane layer 202 by thermo-bonding. In alternative embodiments, the top film layer 206 may be a polyurethane drape coupled to the hydrophilic membrane layer 202 with an adhesive.

The top film layer 206 facilitates evaporation of fluid therethrough from the superabsorbent deposits 204 to the environment. The high moisture vapor transfer rate of the top film layer 206 provides for a high rate of evaporation therethrough. Additionally, because the top film layer 206 is elastic and expands with the superabsorbent deposits 204, the superabsorbent deposits 204 may abut the top film layer 206 at substantially all times, facilitating evaporation of fluid from the superabsorbent deposits 204 through the top film layer 206.

The second superabsorbent laminate 200 is thereby configured to draw fluid through the hydrophilic membrane layer 202, absorb that fluid with the superabsorbent deposits 204, allow the superabsorbent deposits 204 to swell to a substantially maximum volume, and facilitate evaporation of the fluid through the top film layer 206. The second superabsorbent laminate 200 may thereby manage a large amount of fluid exuded from a wound.

The second superabsorbent laminate 200 may be used as a stand-alone dressing, i.e., a dressing consisting of substantially only the hydrophilic membrane layer 202, the superabsorbent deposits 204, and the top film layer 206. In some such embodiments, the hydrophilic membrane layer 202 is thermo-bonded to the top film layer 206. In some such embodiments, an adhesive is included to couple the hydrophilic membrane layer 202, the superabsorbent deposits 204, and the top film layer 206 together. In some such embodiments, the second superabsorbent laminate 200 also includes an adhesive border that allows the second superabsorbent laminate 200 to be adhered to a patient at a wound on the patient. The second superabsorbent laminate 200 may be highly conformable to a wound, highly articulable, and non-bulky as compared to other available dressings.

In other cases, the second superabsorbent laminate 200 may be incorporated into a dressing having additional layers, for example a manifolding layer under the hydrophilic membrane layer 202 that facilitates the application of negative pressure to a wound bed. In such embodiments, the second superabsorbent laminate 200 may include fenestrations, perforations, channels, etc. therethrough such that the second superabsorbent laminate 200 is pneumatically permeable. Such channels are preferably located at least 3 millimeters from the nearest superabsorbent deposit 204.

Referring now to FIG. 3, a third superabsorbent laminate 300 is shown, according to an exemplary embodiment. The third superabsorbent laminate 300 includes a first film layer 302, multiple superabsorbent deposits 304 positioned on the first film layer 302, a second film layer 306, and a fusible fiber layer 308 that couples the first film layer 302 to the second film layer 306. The superabsorbent deposits 304 are positioned between the first film layer 302 and the second film layer 306. In some embodiments, the first film layer 302 may be the same as or similar to the film layer 102 of FIG. 1 while the superabsorbent deposits 304 may be the same as or similar to the superabsorbent deposits 104 of FIG. 1, for example such that the third superabsorbent laminate 300 includes the first superabsorbent laminate 100.

The first film layer 302 may a PVdF film, for example a hydrophilic microporous film as described with reference to the film layer 102 of FIG. 1. In some embodiments, the second film layer 306 is made of the same or similar material(s) as the first film layer 302. In other embodiments, the second film layer 306 includes one or more different materials. For example, the second film layer 306 may be an elastic film with a high moisture vapor transfer rate as described with reference to the top film layer 206 of FIG. 2. As another example, in some embodiments the second film layer 306 is a polyurethane drape, for example a substantially air-tight drape that is sealable over a wound bed and coupleable to a pump to allow the pump to draw a negative pressure at the wound bed.

The fusible fiber layer 308 may include any fibrous material fusible to the first film layer 302 and the second film layer 306. The fusible fiber layer 308 may be porous and/or woven, allowing the flow of fluid therethrough, and, in some embodiments, allowing the superabsorbent deposits 304 to expand (migrate, extend) through the fusible fiber layer 308. The superabsorbent deposits 304 may thereby be allowed to expand laterally within a space between the first film layer 302 and the second film layer 306.

As described in detail with reference to the second superabsorbent laminate 200, the third superabsorbent laminate 300 may be used as a stand-alone dressing consisting of substantially only the first film layer 302, the superabsorbent deposits 304, the fusible fiber layer 308, and the second film layer 306. In other cases, the third superabsorbent laminate 300 is used with other layers to form a dressing.

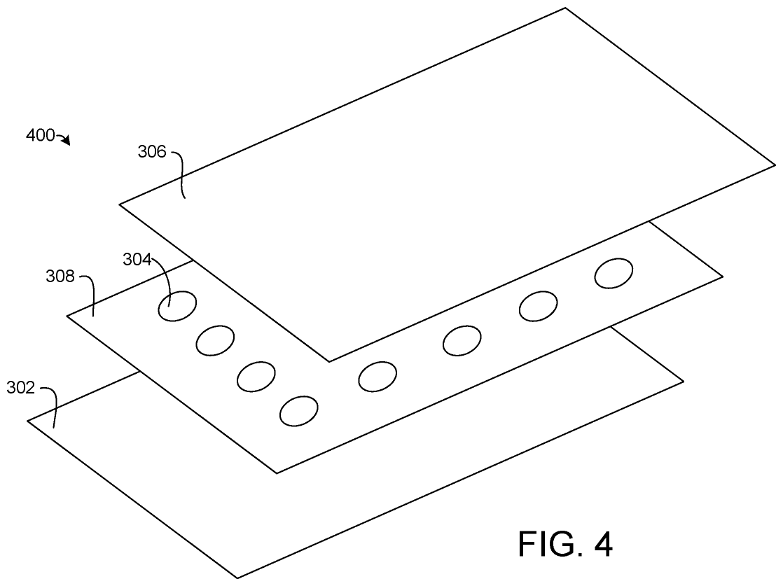

Referring now to FIG. 4, a fourth superabsorbent laminate 400 is shown, according to an exemplary embodiment. The fourth superabsorbent laminate 400 includes the first film layer 302, the fusible fiber layer 308, the superabsorbent deposits 304, and the second film layer 306 as in the third superabsorbent laminate 300 of FIG. 3. However, while superabsorbent deposits 304 are printed/deposited on the first film layer 302 in the third superabsorbent laminate 300, FIG. 4 shows that in the fourth superabsorbent laminate 400 the superabsorbent deposits 304 may be printed onto the fusible fiber layer 308. Printing the superabsorbent deposits 304 onto the fusible fiber layer 308 may facilitate integration of the superabsorbent deposits 304 into the fusible fiber layer 308, for example to encourage expansion of the superabsorbent deposits 304 into gaps (pores, opening, etc.) of the fusible fiber layer 308. It should be understood that various manufacturing processes may be facilitated by printing the superabsorbent deposits 304 onto the fusible fiber layer 308 as in FIG. 4 and/or onto the first film layer 302 as in FIG. 3. Like the third superabsorbent laminate 300, the fourth superabsorbent laminate 400 may be used as a stand-alone dressing consisting of substantially only the first film layer 302, the fusible fiber layer 308, the superabsorbent deposits 304, and the second film layer 306, or may be included with a dressing having one or more additional layers or features.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above. All such variations are within the scope of the disclosure.

What is claimed is:

1. A dressing consisting substantially of:
a hydrophilic membrane layer;
a superabsorbent material positioned on the hydrophilic membrane layer; and
a film layer coupled to the hydrophilic membrane layer and configured to confine the superabsorbent material between the film layer and the hydrophilic membrane layer, the film layer having a high moisture vapor transfer rate.

2. The dressing of claim 1, wherein the superabsorbent material is configured to expand when the superabsorbent material absorbs fluid.

3. The dressing of claim 2, wherein the film layer is elastic and configured to stretch to allow the superabsorbent material to expand towards the film layer.

4. The dressing of claim 3, wherein the hydrophilic membrane layer is configured to substantially restrict expansion of superabsorbent material towards the hydrophilic membrane layer.

5. The dressing of claim 1, wherein the superabsorbent material comprises a plurality of superabsorbent deposits separated from one another.

6. The dressing of claim 5, wherein the plurality of superabsorbent deposits are formed by printing a slurry of the superabsorbent material in a pattern on the hydrophilic membrane layer.

7. The dressing of claim 5, wherein the plurality of superabsorbent deposits are formed by printing a paste in a pattern on the hydrophilic membrane layer, the paste comprising the superabsorbent material and isopropyl alcohol.

8. The dressing of claim 1, wherein the hydrophilic membrane layer is thermo-bonded to the high moisture vapor transfer rate layer and is configured to contact a wound and substantially prevent adherence of the dressing to the wound.

9. The dressing of claim 8, wherein the hydrophilic membrane layer facilitates the flow of fluid from the wound to the superabsorbent material and the film layer facilitates evaporation of fluid from the superabsorbent material, and the hydrophilic membrane layer or the film layer comprises an adhesive border coupleable to a periwound area around the wound.

10. A dressing, comprising:
a first film layer;
a second film layer coupled to the first film layer;
a superabsorbent material positioned between the first film layer and the second film layer; and
a fusible fiber bonding the first film layer to the second film layer and securing the superabsorbent material therebetween.

11. The dressing of claim 10, wherein the first film layer and the second film layer comprise a hydrophilic microporous film configured to allow fluid to flow therethrough and substantially prevent the superabsorbent material from flowing therethrough and to substantially prevent the flow of bacteria therethrough.

12. The dressing of claim 11, wherein the microporous film has a thickness of approximately 125 microns and comprises perforations having diameters between approximately 0.1 microns and 0.2 microns.

13. The dressing of claim 10, wherein the first film layer is a microporous film layer and the second film layer is a polyurethane drape.

14. The dressing of claim 13, wherein the dressing is sealable over a wound bed and coupled to a pump, the pump configured to draw a negative pressure at the wound bed.

15. A method for manufacturing a dressing, comprising:
manufacturing a superabsorbent laminate by:
printing a plurality of superabsorbent deposits on a first film layer; and
coupling the first film layer to a second film layer to substantially confine the superabsorbent deposits between the first film layer and the second film layer; and
coupling the superabsorbent laminate to at least one of a wound contact layer, an absorbent layer, a manifolding layer, or an antimicrobial layer.

16. The method of claim 15, comprising forming the first film layer from a microporous film configured to allow water to flow therethrough and substantially prevent the superabsorbent material from flowing therethrough.

17. The method of claim 15, comprising:
forming the first film layer from a hydrophilic membrane material; and
forming the second film layer from an elastic material having a high moisture vapor transfer rate.

18. The method of claim 15, comprising:
providing a pump and a tube;
preparing the dressing to be fluidly coupled to the pump via the tube, the pump operable to draw a negative pressure at the dressing; and
creating a channel through the superabsorbent laminate to facilitate the flow of air therethrough.

19. The method of claim 15, wherein printing the plurality of superabsorbent deposits on the first film layer comprises depositing a slurry of superabsorbent material in a pattern on the first film layer.

20. The method of claim 15, wherein printing the plurality of superabsorbent deposits on the first film layer comprises depositing a paste in a pattern on the first film layer, the paste comprising the superabsorbent material and isopropyl alcohol.
